(19) United States
(12) Patent Application Publication
(10) Pub. No.: US 2025/0295984 A1
(43) Pub. Date: Sep. 25, 2025

(54) MULTI-SENSORY MAGNETIZED PUZZLE FOR COGNITIVE STIMULATION AND DEMENTIA CARE

(71) Applicant: Faheem Chanda, Oakland, CA (US)

(72) Inventor: Faheem Chanda, Oakland, CA (US)

(21) Appl. No.: 19/084,982

(22) Filed: Mar. 20, 2025

Related U.S. Application Data
(60) Provisional application No. 63/569,128, filed on Mar. 23, 2024.

(51) Int. Cl. A63F 9/12; A61M 21/00

(52) U.S. Cl. CPC A63F 9/1208; A61M 21/00; A61M 2021/0022; A61M 2021/0027; A61M 2021/0044; A63F 2009/1212

(57) ABSTRACT

A multi-sensory cognitive engagement puzzle device for stress reduction, mindfulness, and dementia therapy is disclosed. The device includes a three-dimensional geometric puzzle structure composed of interlocking posts, wherein each post comprises a plurality of interlocking puzzle pieces, wherein the puzzle pieces comprising center puzzle pieces, middle puzzle pieces, external puzzle pieces, wherein each puzzle piece having protrusions, indented sections, grooves configured along at least one of protrusions and indented sections, and magnets, wherein the indented sections are configured to form a continuous outer surface when fully assembled to form the post, and wherein magnets are integrated into grooves of either protrusions and indented sections and configured to slide and lock into, during assembly, either protrusions and indented sections and along each post and between adjacent posts. The surface of each puzzle piece comprises textures distinct to each other to provide distinct tactile sensations.

### CROSS-REFERENCE TO RELATED APPLICATIONS

This application claims the benefit of U.S. Provisional Patent Application No. 63/569,128, filed on Mar. 23, 2024, the entire disclosure of which is incorporated herein by reference.

### FIELD OF THE INVENTION

The present invention relates to a cognitive engagement device, and more particularly to multi-sensory magnetized puzzles designed for stress reduction, mindfulness, and dementia therapy.

### BACKGROUND OF THE INVENTION

Conventional wellness paradigms often overlook the underlying causes of digital-related stress, necessitating innovative interventions that transcend traditional human modalities. In today's modern workplaces, work-related stress and burnout are pervasive issues that have resulted in decreased productivity and increased turnover rates. Despite acknowledging these challenges, current well-being initiatives often overlook the importance of providing employees with tools for relaxation and mindfulness outside of work hours. Additionally, they fail to address the intricate relationship between digital stimuli and employee well-being, leaving employees susceptible to the adverse effects of digital overload. This oversight perpetuates the cycle of stress, impeding employees' ability to fully recharge and perform at their best.

Most corporate wellness programs rely on meditation apps and generic relaxation techniques, which lack physical, cognitive and multi-sensory engagement-either as an individual modality or in combination of multiple modalities. Some examples of these programs include meditation applications, access to massages, access to gyms and occasional seminars to ease the burden of stress. The aforementioned approaches have many limitations including a lack of effectiveness, continuity, and are at times effectively digital solutions-which further exacerbates the problem. Furthermore, despite widespread recognition of the problem, existing corporate wellness initiatives often fail to effectively address burnout, largely due to their time bound, passive nature and lack of sensory engagement. For example, while beneficial for some, digital mindfulness apps often require screen interaction, which can further contribute to digital fatigue rather than alleviate it and these tools lack deeper human sensory engagement. Another common corporate wellness solution is access to a workplace relaxation space. Workplace relaxation spaces (e.g., game rooms, quiet zones) can be underused or ineffective, as they lack structured engagement and do not integrate cognitive restoration techniques.

Alzheimer's disease and related dementias pose significant societal and economic challenges, with millions affected globally. Statistics indicate that approximately 10.8% of individuals aged 75 and above are living with Alzheimer's disease. Separately, current interventions for Alzheimer's disease and related dementias predominantly emphasize pharmacological treatments, such as cholinesterase inhibitors and monoclonal antibody therapies, aimed at managing symptoms or slowing disease progression. However, these approaches often come with significant limitations, including variable efficacy, potential side effects, and high costs, making them inaccessible or unsuitable for all patients.

In contrast, non-pharmacological interventions, such as cognitive stimulation, sensory engagement, and structured social interaction, offer a holistic and complementary approach to dementia care. Despite growing evidence supporting their effectiveness in enhancing cognitive function, reducing behavioral symptoms, and improving overall well-being, these methods remain underutilized in standard care protocols. There is a need for an accessible and engaging tool that recognizes the critical role of multi-sensory experiences, problem-solving activities, and therapeutic engagement and that can meaningfully enhance the quality of life for individuals with dementia while supporting caregivers and healthcare providers in delivering comprehensive, patient-centered care.

### BRIEF SUMMARY OF THE INVENTION

It is an object of the present invention to provide a multi-sensory, multi-layered puzzle that enhances cognitive stimulation, problem-solving skills, and spatial reasoning through tactile, auditory, and visual engagement.

It is another object of the present invention to offer corporate users an interactive tool for stress relief, focus enhancement, and mindfulness, promoting relaxation and mental clarity after work.

It is yet another object of the present invention to facilitate social interaction and emotional regulation by enabling collaborative puzzle-solving, particularly for elderly users in dementia therapy.

It is a further object of the present invention to encourage digital detoxification by providing an engaging, screen-free activity that promotes sensory engagement and creative thinking.

It is still another object of the present invention to support work-life balance by offering employees a structured yet flexible decompression method that aids in transitioning from work to leisure effectively.

According to an embodiment a multi-sensory cognitive engagement puzzle device for stress reduction, mindfulness, and dementia therapy is disclosed. The device includes a three-dimensional geometric puzzle structure composed of interlocking posts, wherein each post comprises a plurality of interlocking puzzle pieces, wherein the puzzle pieces comprising center puzzle pieces, middle puzzle pieces, external puzzle pieces, wherein each puzzle piece having protrusions, indented sections, grooves configured along at least one of protrusions and indented sections, and magnets, wherein the indented sections are configured to form a continuous outer surface when fully assembled to form the post, and wherein magnets are integrated into grooves of either protrusions and indented sections and configured to slide and lock into, during assembly, either protrusions and indented sections and along each post and between adjacent posts. The surface of each puzzle piece comprises textures distinct to each other to provide distinct tactile sensations and a plurality of color-coded sections configured to provide visual distinction and stimulate cognitive function.

### BRIEF DESCRIPTION OF DRAWINGS

The present invention will become clearly understood to those of ordinary skill in the art when descriptions of exemplary embodiments thereof are read with reference to the accompanying drawings.

FIG. 1 depicts a perspective view of a fully assembled puzzle device in its spherical configuration.

FIG. 2 depicts an alternative non-spherical configuration of the puzzle device, demonstrating how the same component pieces can be reconfigured to form different geometric shapes.

FIG. 3 depicts a detailed view of a middle puzzle piece of the indented sections for magnets, alignment grooves, contoured edges, and wave-patterned texture on the exterior surface.

FIG. 4 depicts a center puzzle piece with protrusions for magnet installation.

FIG. 5 is an assembly view showing the alignment and interconnection of center, middle, and external puzzle pieces.

FIG. 6 is a detailed view of an external puzzle piece, highlighting its contoured edges and sliding grooves.

FIG. 7 illustrates a fully assembled post comprising one center, a number of middle, and a number of external interlocked puzzle pieces.

### DETAILED DESCRIPTION OF THE INVENTION

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. For a more complete understanding of the present invention, reference is now made to the following descriptions taken in conjunction with the accompanying drawings.

The intersection of wellness, cognitive health, and sensory engagement provides an opportunity for innovation in both corporate and therapeutic settings. Traditional approaches fail to address the multifaceted nature of human cognition, with corporate wellness programs relying heavily on digital solutions that can exacerbate screen fatigue, while dementia care remains dominated by pharmacological interventions despite their limitations in efficacy, side effects, and cost.

A multi-sensory, multi-layered magnetized puzzle device described in the present invention combines tactile, auditory, visual, and cognitive stimulation in a single device. Its configurable difficulty system makes it adaptable for diverse users, from corporate professionals seeking stress reduction to elderly individuals requiring cognitive maintenance therapy, while its physical nature provides a much-needed break from digital stimulation that characterizes modern workplaces.

This invention has significant advantages through sensory engagement that activates multiple neural pathways, adaptive cognitive stimulation that can be customized to individual abilities, and the social interaction potential that fosters connection between caregivers and patients or colleagues in workplace settings. By integrating these elements into a tangible, engaging tool, the device addresses critical gaps in both corporate wellness and dementia care, while recognizing the complex interplay between sensory experience, cognitive function, and emotional regulation.

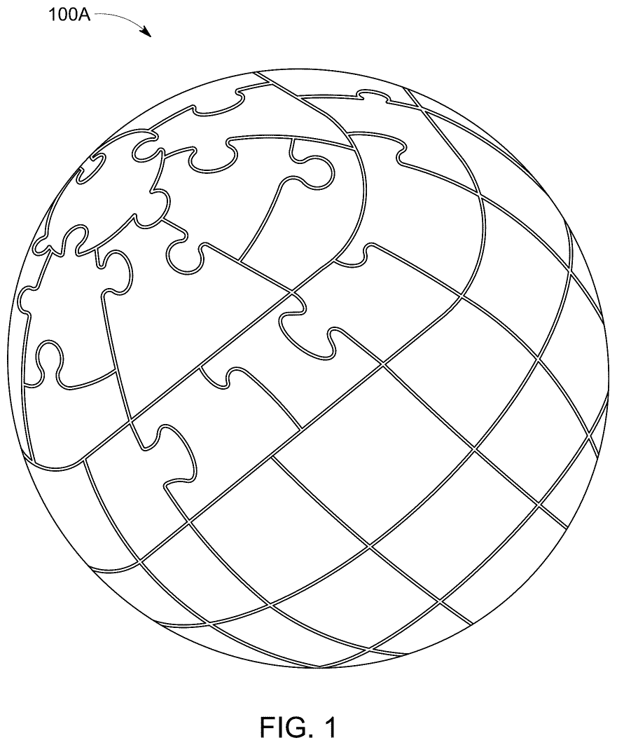

Referring to FIG. 1, an exemplary embodiment of a multi-sensory, multi-layered magnetized puzzle device 100A (hereinafter referred to as the “device” or “puzzle”) is disclosed. The device 100A is depicted as an assembled device 100A in a spherical configuration according to this embodiment. In an exemplary aspect, the device 100A is configured as a three-dimensional geometric puzzle device 100A which includes a plurality of interlocking posts 101A-101N, wherein each post comprises a plurality of interlocking puzzle pieces. In some aspects, the device 100A incorporates a plurality of colors, textures, and assembly elements configured to engage multiple senses simultaneously, including tactile feedback, magnetic interactions, hand-eye coordination, and visual stimulation through a mix of colors.

While FIG. 1 illustrates a spherical configuration, it should be understood that the puzzle structure is configurable to form various three-dimensional shapes including cubic, pyramidal, or abstract geometric forms, depending on the arrangement and design of the component posts 101A-101N and pieces.

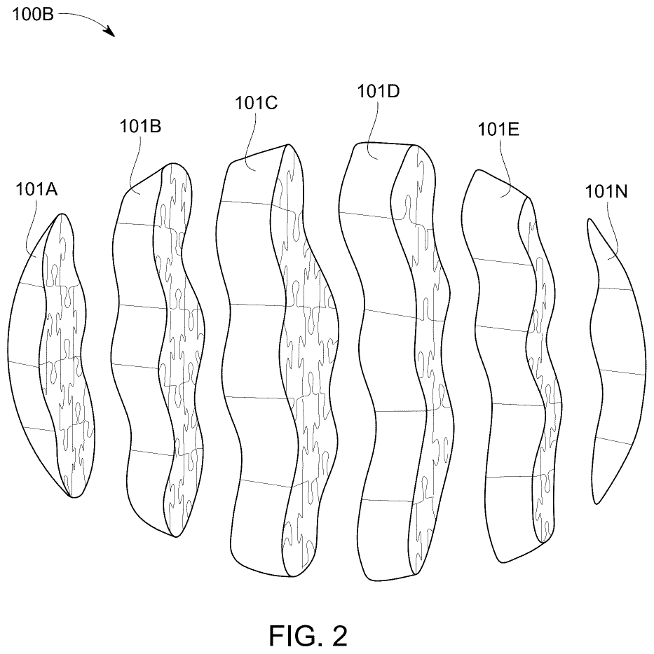

FIG. 2 illustrates the puzzle device in a disassembled state, showing the individual posts 101A-101N that make up the complete structure. The puzzle consists of multiple vertical posts, including the left edge post 101A, left middle post 101B, left inner post 101C, right inner post 101D, right middle post 101E, and right edge post 101N, all of which have a non-spherical configuration. Each post is composed of interlocking puzzle pieces that can be assembled by sliding magnetized sections into place, offering an engaging and guided assembly experience. In some aspects, the exterior of these posts may feature different textures, colors, and dimensions to enhance sensory interaction. The assembly process introduces a multi-dimensional challenge, requiring spatial reasoning and fine motor skills to construct the puzzle.

In various embodiments, posts 101A-101N may vary in length, curvature, or angularity to accommodate different geometric configurations. Some posts 101A-101N may feature vertical and lateral curves or angles that increase assembly complexity, while also enabling the formation of various structural outcomes when the posts 101A-101N are arranged in different orientations.

In an exemplary aspect, the puzzle comprises three primary types of puzzle pieces: a number of center pieces, a number of middle pieces 103, and a number of external pieces, each configured with distinct structural and functional features.

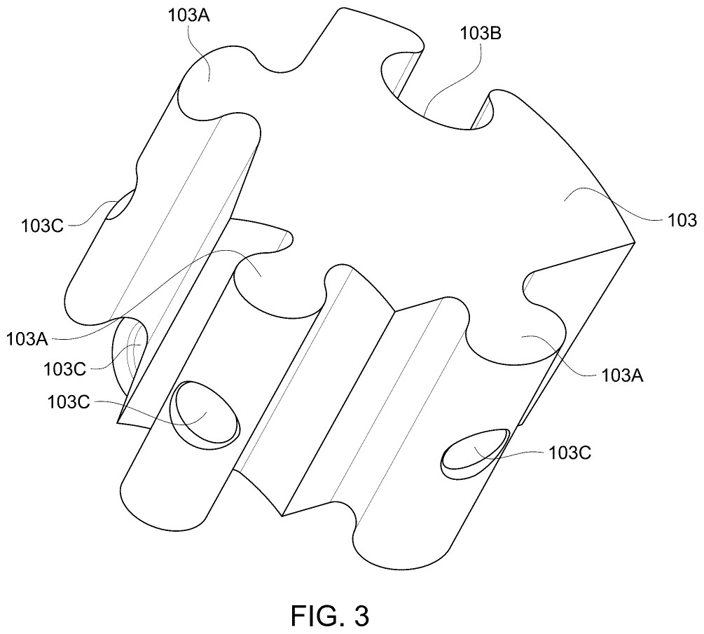

Referring to FIG. 3, the middle puzzle piece 103 includes one or more protrusions 103A, one or more grooves 103C, one or more magnets (not shown) and one or more indented sections 103B. The one or more grooves 103C are configured along at least one of indented sections 103B or protrusions 103A. In some aspects, the grooves 103C are configured along the protrusions 103A for integrating magnets that allow puzzle pieces to slide into and lock with adjacent puzzle pieces, i.e. magnets of each puzzle piece slide and lock into grooves 103C of indented sections 103B along place along each post and between adjacent posts 101A-101N. In some other aspects, the grooves 103C are configured along the indented sections 103B for integrating magnets that allow puzzle pieces to slide into and lock with adjacent puzzle pieces, i.e. magnets of each puzzle piece slide and lock into grooves 103C of protrusions 103A, during assembly by the user, along each post and between adjacent posts 101A-101N. The protrusions 103A, grooves 103C and indented sections 103B provide a secure connection between adjacent puzzle pieces via the magnets. The middle pieces 103 serve as connective elements between the center and external pieces.

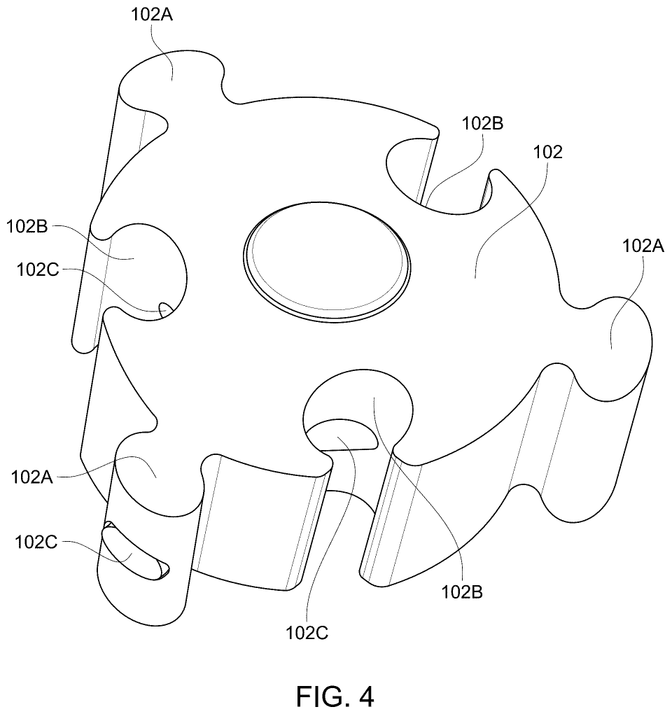

As shown in FIG. 4, the center puzzle piece 102 features a jigsaw-inspired design that enhances user engagement through haptic and magnetic interactions. In an exemplary aspect, the center puzzle piece 102 includes one or more protrusions 102A, one or more grooves 102C, one or more magnets (not shown) and one or more indented sections 102B. The one or more grooves 102C are configured along at least one of indented sections 102B or protrusions 102A. In one aspect, the magnets are configured to be installed into the grooves 102C of indented sections 102B for facilitating secure sliding assembly. In another aspect, the magnets are configured to be installed into the grooves 102C of protrusions 102A. The magnets are arranged in a predetermined pattern into grooves 102C to enhance haptic engagement. In one aspect the indented sections 102B are configured to receive magnets which are attached to the grooves 102C, during assembly by the user, enabling smooth connection with adjacent pieces. In another aspect the grooves 102C are configured to receive magnets which are attached to the indented sections 102B, during assembly by the user, enabling smooth connection with adjacent pieces.

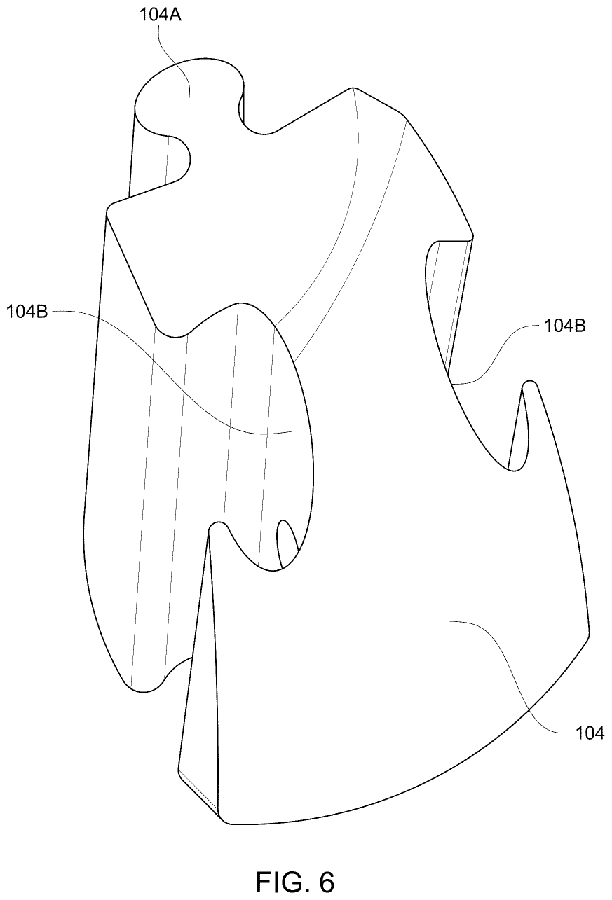

Referring to FIG. 6, an external puzzle piece 104 is depicted which includes one or more protrusions 104A, one or more grooves 104C, one or more magnets (not shown) and one or more indented sections 104B. The one or more grooves 104C are configured along at least one of indented sections 104B or protrusions 104A. The indented section 104B forms contoured edges on a continuous outer surface when fully assembled, which may be spherical or another geometric form depending on the configuration. Each external piece interlocks with adjacent pieces to maintain the structural integrity of the completed puzzle. In one aspect, magnets are integrated into the groove 104C of indented sections 104B and then locked with grooves 104B of protrusions 104A, during assembly, for secure attachment with adjacent puzzle pieces. In another aspect, magnets are integrated into the groove 104C of protrusions 104A and then locked with grooves 104B of indented sections 104B, during assembly, for secure attachment with adjacent puzzle pieces. The protrusions 104A, groove 104C and indented section 104B are configured to integrate the magnets to guide the assembly process and maintain structural integrity of the completed form.

In an exemplary aspect, the indented sections 102B, 103B, 104B of each puzzle piece are configured to form a continuous outer surface when fully assembled to form the post. In various aspects, the term “indented sections” are also referred to as indented contours or contoured edges which are configured around the edges of each piece. In various aspects, the term “grooves” are also referred to as sliding grooves, sliding slots or alignment grooves. In various aspects, the term “magnets” are also referred to as magnetic connectors or magnetic elements.

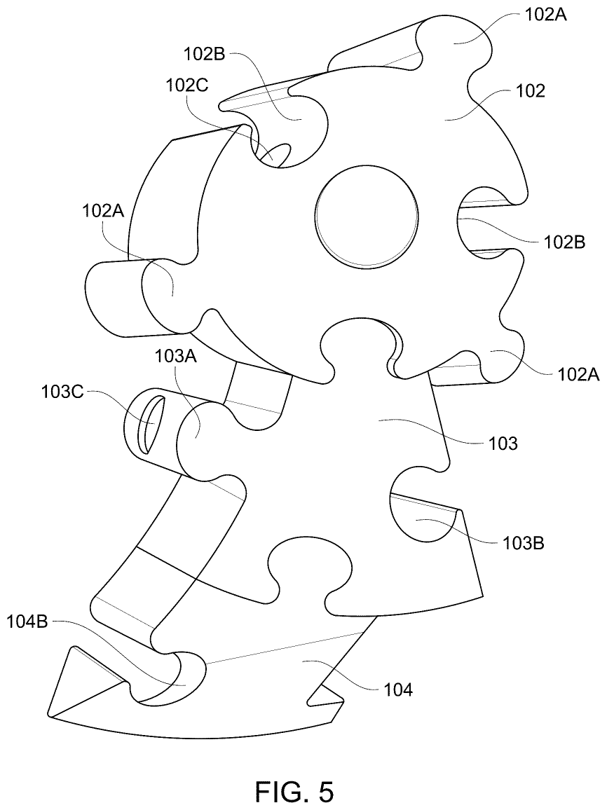

FIG. 5 depicts the alignment and interconnection of the center, middle, and external puzzle pieces to provide proper structural assembly. This figure illustrates how the different pieces interlock through a sliding mechanism (i.e. via protrusions 102A, 103A, 104A, grooves 102C, 103C, 104C and indentations 102B, 103B, 104B) and magnetic connections to create a secure structural unit.

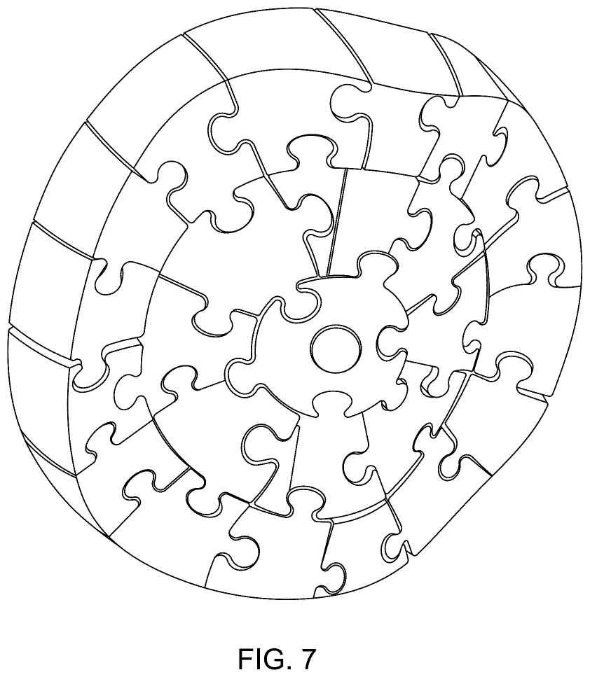

As illustrated in FIG. 7, each assembled post comprises multiple interlocking puzzle pieces that may include a variety of shapes, magnets, textures, and colors. Posts 101A-101N include lateral and vertical curves, which contribute to increasing the complexity of the puzzle assembly. The complete puzzle integrates haptic, tactile, and magnetic interactions, enhancing cognitive engagement and mindfulness. When fully assembled, the posts 101A-101N interlock with one another to form the complete geometric structure, thereby creating a cohesive three-dimensional puzzle 100A or 100B that challenges spatial reasoning and problem-solving abilities.

The magnetic elements are configured to produce an audible click when puzzle pieces are correctly aligned, thus providing auditory reinforcement and sensory feedback. The magnets create secure connections between adjacent puzzle pieces through indentations housing embedded magnets, and guide the sliding assembly process through magnetic attraction and repulsion. In some aspects, the magnets facilitate guided alignment and provide tactile feedback during assembly, thus enhancing the sensory experience of solving the puzzle 100A or 100B. The sliding assembly mechanism, which includes alignment grooves, allows magnetized puzzle pieces to slide and lock into place along each post and between adjacent posts 101A-101N.

In various embodiments, the puzzle pieces 100A or 100B comprise a combination of materials, which may include wood, metal, and polymer components. Each material provides different tactile properties including variations in texture, temperature, and weight, thus enhancing the multi-sensory nature of the puzzle 100A or 100B. In some embodiments, the surface of each puzzle piece comprises textures distinct from other pieces to provide varied tactile sensations. Additionally, the pieces incorporate color-coded sections designed to provide visual distinction and stimulate cognitive function. Further, the puzzle 100A or 100B incorporates a configurable difficulty system wherein posts 101A-101N can be arranged in different configurations to increase or decrease complexity. Additional puzzle pieces can be incorporated to expand the challenge level, and alternative assembly patterns can be introduced for extended cognitive engagement.

Example embodiment of arranging the puzzle pieces is disclosed herein. The assembly begins with the center puzzle pieces, which serve as the structural foundation for each post. These center pieces must be arranged first in their approximate final orientation, creating the framework of the three-dimensional structure. The arrangement of these center pieces determines whether the completed puzzle forms a spherical, cubic, pyramidal, or abstract geometric configuration.

Following the placement of center pieces, middle puzzle pieces are connected in a specific sequence. A first middle piece slides onto one end of each center piece, with the magnets guiding proper alignment. The user must align the protrusions of the center piece with the corresponding indented sections of the middle piece until an audible click confirms correct placement. This initial connection establishes the primary axis of each post. The second middle piece is then attached to the opposite end of the center piece, completing the basic post structure and establishing its curvature or angularity.

Once the center and middle pieces are connected for each post, the external puzzle pieces are attached to complete the post assembly. These external pieces must be added in a specific order, beginning with attachment to the middle piece at one end of the post, followed by the middle piece at the opposite end. The sequence of external piece attachment influences the final shape and stability of the assembled puzzle.

The posts themselves must be connected in a specific sequence to form the complete three-dimensional structure. Beginning with two initial posts connected via their external pieces, additional posts are consequently added to the growing structure. The magnetic guidance assists users in finding correct post-to-post connections, with the auditory feedback confirming proper alignment. This progressive assembly sequence creates a scaffolded cognitive challenge that builds spatial reasoning skills while providing continual sensory feedback through tactile, visual, and auditory channels.

### Example Implementation of the Present Invention

For instance, the puzzle comprises twelve interlocking posts arranged to form a spherical structure approximately 8 inches in diameter when fully assembled. The complete structure weighs approximately 1.2 pounds, providing substantial tactile feedback while remaining lightweight enough for extended manipulation.

Each post within the puzzle structure includes a curved design with angles varying between 30° and 45°, thus enabling the formation of the spherical configuration when properly assembled. Posts vary in length from 6 to 8 inches, with this variation contributing to the spatial reasoning challenge presented by the puzzle. The curvature and varied lengths of the posts require users to engage spatial cognition skills during assembly, enhancing the cognitive benefits of the device.

Each post comprises 13 and up to, but not limited to 25 interlocking puzzle pieces. Center puzzle pieces measure approximately 1.2×1.2×1 inches (3×3×2.5 cm) and are primarily constructed from polymers, wood or metal. Each center piece contains eight neodymium magnets with dimensions of 5 mm diameter and 2 mm thickness, arranged in a diamond pattern to facilitate proper alignment with adjacent pieces.

The center puzzle pieces feature jigsaw-inspired protrusions on both ends that correspond to recessed areas on the middle pieces, thus enabling secure connections while challenging fine motor skills. The outer surface of these pieces incorporates ridge patterns contrasted with smooth indentations, thus creating a tactile feedback. These pieces are color-coded using primary colors (red, blue, yellow) with subtle gold accent markings to provide visual orientation cues during assembly.

Middle puzzle pieces, measuring 1.25×1.25×1.5 inches, are constructed from polymers, wood or metal. Each middle piece contains 4 to 6 neodymium magnets positioned to create secure connections with both center and external pieces. The exterior surface of the middle pieces may include wave patterns of varying depths, providing distinctive tactile feedback that differs from the center pieces. These middle components are color-coded using secondary colors (green, orange, purple or a variety of colors) with possible silver accent markings, thus creating a visual progression from the primary-colored center pieces.

External puzzle pieces, with dimensions approximately of 1×1×0.75 inches, are constructed from polymer, wood or metals. These pieces incorporate up to, but not limited to 5 neodymium magnets each, positioned to align with the magnets in the middle pieces. The exterior surfaces of these pieces feature a variety of tactile textures including ribbed, dotted, and smooth areas. When correctly assembled, the external pieces form a continuous spherical outer surface.

In this example, N52 grade neodymium magnets are used, totaling up to 500 throughout the complete puzzle. The magnets are arranged with specific polarity orientations to prevent incorrect assembly while guiding users toward the correct configuration. When puzzle pieces align correctly, the magnetic connection produces a distinctive audible “click”, thereby providing auditory reinforcement of successful assembly steps. The magnetic force between connecting pieces ranges from 0.5 to 0.8 kg pull force, thus creating connections that are secure yet can be overcome with deliberate manipulation, and thus enhancing fine motor engagement.

In this example, the puzzle implements a configurable difficulty system. In the standard configuration, posts are arranged symmetrically with matching colors indicating connecting pieces, which is suitable for beginners. For intermediate difficulty, posts are arranged in mixed orientations requiring enhanced spatial reasoning, with a complementary color system that requires understanding of color relationships. The advanced configuration features asymmetrical post arrangements and the introduction of challenge pieces with multiple possible connections, which significantly increases the cognitive demands.

The puzzle device is adaptable as it is suitable for users of varying skill levels, from beginners to advanced puzzle enthusiasts, and allows for progressive difficulty adjustment for therapeutic applications. Advantageously, the puzzle device provides multiple therapeutic and wellness benefits across different applications and user groups. For corporate users, the puzzle serves as a stress reduction tool that allows employees to decompress and refocus. The multi-sensory engagement encourages mindfulness and present-moment awareness, providing a mental break from work-related stress. Regular interaction with the puzzle may improve focus, creativity, and problem-solving abilities in the workplace.

For elderly users, particularly those experiencing cognitive decline or at risk for dementia, the puzzle provides cognitive stimulation through its combination of spatial reasoning, memory, and fine motor skill challenges. The multi-sensory nature of the puzzle engages multiple neural pathways simultaneously, thus potentially helping to maintain cognitive function and delay symptom progression.

The collaborative aspect of the puzzle also encourages social interaction, which is beneficial for emotional well-being and cognitive health. Caregivers and patients can work together on the puzzle, fostering connection and communication. The rhythmic, structured nature of assembling the puzzle can have a calming effect, thus helping users regulate emotions during periods of anxiety or agitation. The tactile engagement and focused attention required help redirect negative thoughts and promote a state of flow, which has been associated with improved mood and reduced stress.

The multi-sensory puzzle device combines physical, cognitive, and sensory elements to create an engaging therapeutic tool with applications ranging from corporate wellness programs to dementia care. Its adaptable design allows for use across varying skill levels and therapeutic needs, while its multi-sensory nature maximizes cognitive engagement and benefits.

It will finally be understood that the disclosed embodiments are presently preferred examples of how to make and use the claimed invention, and are intended to be explanatory rather than limiting the scope of the invention as defined by the claims below. Reasonable variations and modifications of the illustrated examples in the foregoing written specification and drawings are possible without departing from the scope of the invention as defined in the claim below. It should further be understood that to the extent the term “invention” is used in the written specification, it is not to be construed as a limited term as to number of claimed or disclosed inventions or the scope of any such invention, but as a term which has long been conveniently and widely used to describe new and useful improvements in technology. The scope of the invention supported by the above disclosure should accordingly be construed within the scope of what it teaches and suggests to those skilled in the art, and within the scope of any claims that the above disclosure supports. The scope of the invention is accordingly defined by the following claims.

This application is intended to cover any adaptations or variations of the present invention. Therefore, it is manifestly intended that this invention be limited only by the claims and the equivalents thereof.

1. A multi-sensory cognitive engagement puzzle device, comprising:
a three-dimensional geometric puzzle structure composed of a plurality of interlocking posts, wherein each post comprises a plurality of interlocking puzzle pieces;
wherein the plurality of puzzle pieces comprising:
one or more center puzzle pieces, one or more middle puzzle pieces, one or more external puzzle pieces, wherein each puzzle piece having one or more protrusions, one or more indented sections, one or more grooves configured along at least one of protrusions and indented sections, and one or more magnets;
wherein the indented sections are configured to form a continuous outer surface when fully assembled to form the post;
wherein magnets are integrated into grooves of either protrusions and indented sections and configured to slide and lock into, during assembly, either protrusions and indented sections and along each post and between adjacent posts;
wherein surface of each puzzle piece comprises:
textures distinct to each other to provide distinct tactile sensations; and
a plurality of color-coded sections configured to provide visual distinction and stimulate cognitive function.

2. The multi-sensory puzzle device of claim 1, wherein the geometric puzzle structure is configurable to form various three-dimensional shapes including spherical, cubic, pyramidal, or abstract geometric forms.

3. The multi-sensory puzzle device of claim 1, wherein the posts are configurable in different orientations to create various structures.

4. The multi-sensory puzzle device of claim 3, wherein the different orientations of posts creates a multi-dimensional challenge requiring spatial reasoning to solve.

5. The multi-sensory puzzle device of claim 1, wherein each post comprises vertical and lateral curves or angles that increase assembly complexity.

6. The multi-sensory puzzle device of claim 1, wherein posts are configured to interlock with one another to form the complete geometric structure.

7. The multi-sensory puzzle device of claim 1, wherein the posts vary in length, curvature, or angularity to accommodate different geometric configurations.

8. The multi-sensory puzzle device of claim 1, wherein the magnets are configured to:
produce an audible click when puzzle pieces are correctly aligned to provide auditory reinforcement;
create secure connections between adjacent puzzle pieces through indented sections housing embedded magnets; and
guide the sliding assembly process through magnetic attraction and repulsion.

9. The multi-sensory puzzle device of claim 1, wherein the puzzle pieces comprise a combination of wood, metal, and polymer materials, wherein each material provides different tactile properties including variations in texture, temperature, and weight.

10. The multi-sensory puzzle device of claim 1, further comprising a configurable difficulty system wherein:
posts are arranged in different configurations to increase or decrease complexity;
additional puzzle pieces are incorporated to expand the challenge level; and
alternative assembly patterns are introduced for extended cognitive engagement.
